(19) United States
(12) Patent Application Publication
(10) Pub. No.: US 2014/0366891 A1
(43) Pub. Date: Dec. 18, 2014

(54) Easy Steady Oral Protection

(71) Applicant: Zaid Umar Bettis, Englewood, NJ (US)

(72) Inventor: Zaid Umar Bettis, Englewood, NJ (US)

(21) Appl. No.: 13/914,543

(22) Filed: Jun. 13, 2013

(51) Int. Cl. A61F 5/44

(52) U.S. Cl. CPC A61F 5/4408
USPC 128/859

(57) ABSTRACT

“Easy Steady Oral Protection” is designed to aid in the protection against sexually transmitted diseases (STD) and sexually transmitted infections (STI), which can be contracted during oral intercourse, both vaginal and anal. Furthermore, as cases of the Human Papillomavirus (HPV) are increasing with this virus linked to causing cervical cancer in women, it may also be contracted during the act of oral intercourse in heterosexual men, the transgendered, lesbian and /or gay women and linked to throat cancer among non-smoking individuals associated with these groups. This prophylactic device is comprised of latex or polyurethane or polyisoprene or similarly natural rubber material designed and intended to aid in the protection against these infections and viruses when “properly used”.

This prophylactic is intended and designed for use by both male(s) and female(s) during the act or performance of oral intercourse, both vaginal and anal. “Easy Steady Oral Protection” is designed to fit snuggly against the lower portion of the wearers face covering the mouth, extending out just under the nose and extends laterally to meet the “Dimples” of each facial cheek and just under the base of the chin line, secured by 2 (two) straps on each side which is to be placed behind each ear of the wearer. This securing method will hold the “Easy Steady Oral Protection” in place.

“Easy Steady Oral Protection” is designed as a “Rectangular” or “Square Shaped” shaped device made of latex, polyurethane or polyisoprene material or other natural rubber material similarly formed and molded to securely cover the mouth from the upper lip just below the nose (Mustache line), to the bottom of the base of the chin, out to the dimple of each facial cheek. This device is also designed in two sizes to incorporate the difference in facial sizes of both males and females. In the center of “Easy Steady Oral Protection” there is an opening which contains a unique “tongue protrusion” allowing for the tongue to be extended, protracted and ultimately rotated in any desired direction. The wearers tongue is also protected, as this protrusion is part of the of the “Easy Steady Oral Protection” product as a whole. The “tongue protrusion” allows the wearer to achieve deep penetration into the vagina or anal cavity in addition to the use of the mouth and lips for the external pleasuring the of the anal surfaces, vaginal surfaces, vaginal lips, clitoris and associated surface or reachable areas while simultaneously maintaining a protective barrier from the bodily fluids and secretions of each partner. The lip portion and “tongue insert and/or tongue protrusion” of “Easy Steady Oral Protection” has a design which allows for this portion of the product, which is symmetrically positioned to cover the lips and centering the tongue, creates both flexibility and tautness, as the product rest securely about the face and mouth. The flexibility and security of this design feature will permit the wearer to perform as if there were little to no bearer between partners, yet providing the protection against the exchange of bodily fluid. This design is also enhanced by the symmetrically positioned sections which cover the lips, mouth and center the tongue, as this portion of the product is designed to be retracted inward approximately 1″- 2″ (one inch to two inches), thus providing the wearer additional aid in controlling the possible or excessive accumulation of saliva during oral intercourse within the protective device. The wearer will be able to comfortably control and/or swallow excess saliva without removing the product or compromising its protective integrity.

In addition to the universally form fitting design, “Easy Steady Oral Protection” is secured in place to the wearers face and mouth by the use of 2 (two) adjacent ear-traps. These ear-straps are located at the outermost tip of the product and are identified by 2 (two) colored dots, one on each side for each ear-strap. These colored dots also indicate which side of the product is to be worn outward, distinguishing the outer from the inner, insuring the product is worn properly and ready to be used as directed. However, in the event that the device is put on backwards, it is designed so that the protective effectiveness will not be compromised.

“Easy Steady Oral Protection” is a disposable product. It is not to be re-used. It is designed to be used one time and then discarded. Multiple uses of a single “Easy Steady Oral Protection” severely reduce and/or negate its disease and/or virus prevention abilities. Use of lubricants such as petroleum based products or oils are not recommended, as they can cause slippage, breakage, weakening and/or the general malfunction of the product.

### OPERATIONAL INSTRUCTIONS

“Easy Steady Oral Protection” is to be worn as follows:
a. Open packaging and remove “Easy Steady Oral Protection”
b. Identify outer surface by locating the (2) colored dots at the end of each ear strap.
c. Place “Easy Steady Oral Protection” over the mouth centering the tongue protrusion to line up with the wearers tongue.
d. Before securing device to the face, gently blow or exhale into “tongue protrusion”. This action will slightly inflate and/or deploy the tongue section of the “Easy Steady Oral Protection”, thus opening it for the insertion of the tongue.
e. Insert tongue into tongue protrusion and secure snuggly to face. Secure both ear straps.
f. “Easy Steady Oral Protection” is now fully donned and ready for use. It should be noted that excessive snatching and pulling may pull the device away from the face, putting both partners at risk of mixing bodily fluids and increasing the risk of the contraction of virus and/or disease.

### DRAWINGS

Provided are detailed diagrams of “Easy Steady Oral Protection”. These diagrams provide several different views detailing the specific details of the “Easy Steady Oral Protection” product. These views are as follows:

FIG. 1—Front ¾ View

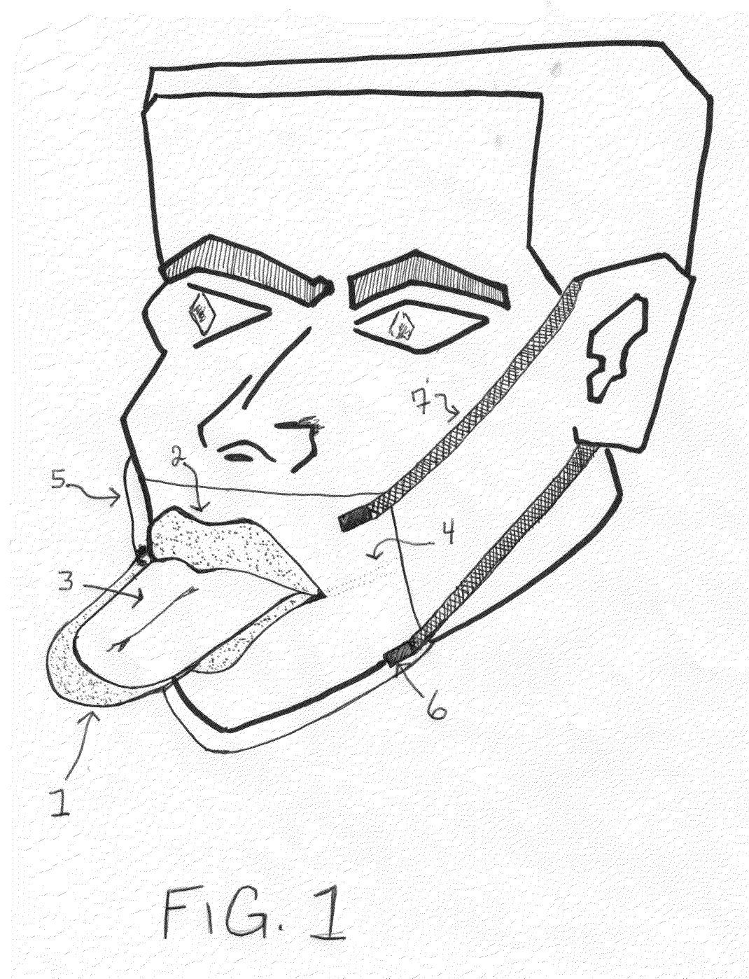

FIG. 2—Side View

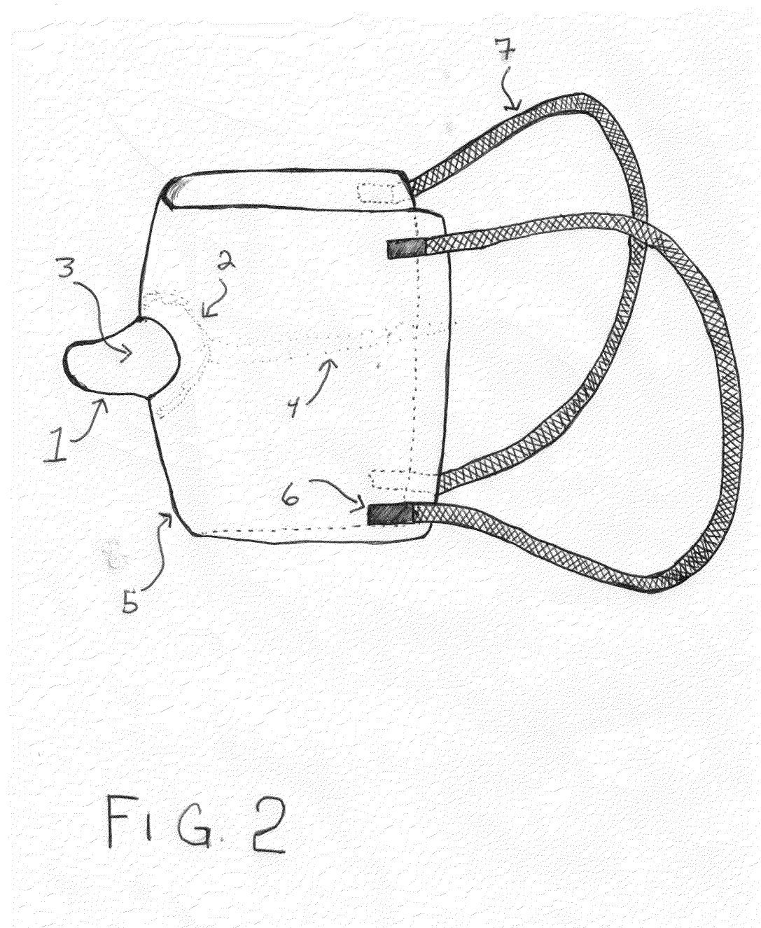

FIG. 3—Rear 3/4 View

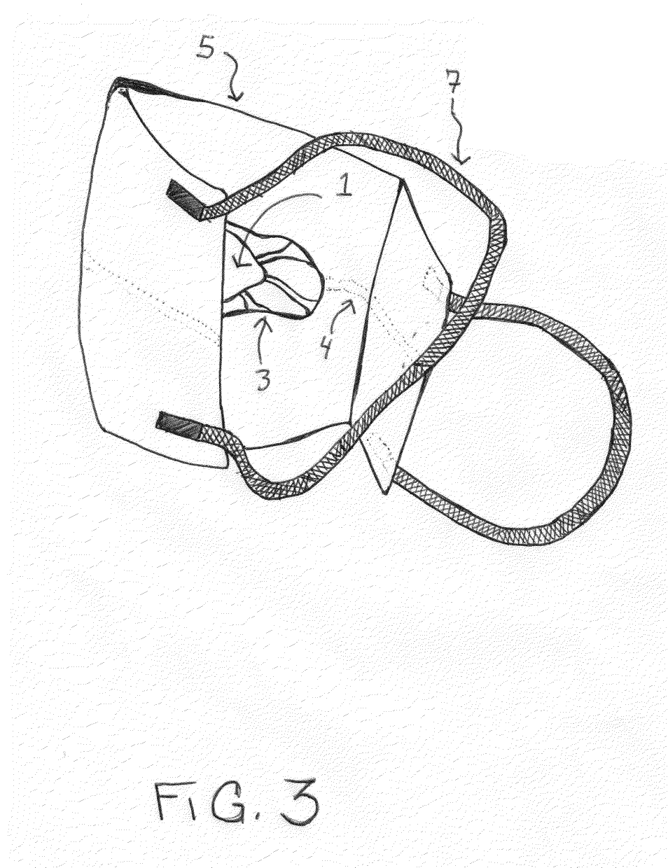

I CLAIM:

1. “Easy Steady Oral Protection” is a flexible, “Rectangular” or “Square” shaped, non-permeable device (#5) designed with the sole task of preventing the transfer of bodily fluids and/or secretions between individuals during oral intercourse.

2. This device (#5) consists of latex, polyurethane, polyisoprene or other natural rubber products, which snuggly covers the face from the “Mustache Line” and/or just under the nose and/or approximately Vs inch below the nose, extending down just under the chin (Vertically) and extending (Horizontally) from the point of each dimple at each point of each cheek.

3. This device consists of a tongue protrusion (#1), dead center of the device.

4. This device contains a unique and universally sized molded lip(#2), both upper and lower for relatively any size wearer, thus allowing for a full range of sensation, opening and closing of the mouth with the “natural” and “comfortable” use of the lips, and “natural flow” during use. This device consists of a universal, upper and lower “Form Fitted Lip Canal Molding” (#2) as a key component of the device which allows for a snug facial fit and enhances the ability to perform using a full range of lip movements and motions without being fully hindered by the barrier.

5. (canceled)

6. (canceled)

7. (canceled)

8. (canceled)
